(19) United States
(12) Patent Application Publication
(10) Pub. No.: US 2021/0001030 A1
(43) Pub. Date: Jan. 7, 2021

(54) PERITONEAL DIALYSIS MACHINE

(71) Applicant: Fresenius Medical Care Deutschland GmbH, Bad Homburg (DE)

(72) Inventors: Peter WABEL, Rosbach (DE); Klaus WOLF, Muedesheim (DE); Stefan WEISS, Bad Homburg (DE)

(73) Assignee: Fresenius Medical Care Deutschland GmbH, Bad Homburg (DE)

(21) Appl. No.: 16/971,432

(22) PCT Filed: Feb. 14, 2019
(86) PCT No.: PCT/EP2019/053685

§ 371 Date: Aug. 20, 2020

(30) Foreign Application Priority Data
Feb. 21, 2018 (DE) 10 2018 103 918.2

(51) Int. Cl. A61M 1/28; A61M 1/16; A61M 1/00; A61M 39/24; A61M 39/28; A61M 39/10

(52) U.S. Cl. CPC A61M 1/282; A61M 1/285; A61M 1/1672; A61M 39/10; A61M 1/0011; A61M 39/24; A61M 39/28; A61M 1/1645

(57) ABSTRACT

The present invention relates to a peritoneal dialysis machine having means for carrying out a peritoneal dialysis treatment, wherein the machine has a drain line which is connected or connectable to the patient catheter and through which consumed dialysis solution is conducted into a drain, wherein the drain line does not open into a drainage bag and wherein the drain line is connected at an opening to a venting line by means of which the venting line can be vented, with a sterile filter being arranged in the drain line upstream of the opening and/or in the venting line and with a cut-off means by means of which the drain line can be cut off at times is provided in the drain line upstream of the opening.

The present invention relates to a peritoneal dialysis machine having means for carrying out a peritoneal dialysis treatment, wherein the machine has a drain line which is directly or indirectly connected or connectable to the patient catheter and through which the consumed dialysis solution is conducted into a drain.

It is customary in the field of peritoneal dialysis to provide the patient with solution bags that are filled with a dialysis solution suitable for the patient. The patient then connects these solution bags to an inflow hose, i.e. to the patient catheter, independently or with the aid of trained medical personnel to fill the peritoneum with the fresh dialysis solution. After the end of the treatment, i.e. after a certain time to take effect has elapsed, the solution is removed from the abdomen of the patient again and is let out into a drainage container or drainage bag, i.e. into a container or bag for receiving the consumed dialysis solution.

It is the underlying object of the present invention to further develop a peritoneal dialysis machine of the initially named kind such that drainage containers or drainage bags can be dispensed with, with it being ensured that a reliable and complete emptying of the drain line is always achieved after the treatment.

This object is achieved by a peritoneal dialysis machine having the features of claim 1. Provision is accordingly made that the drain line does not open into a drainage container or drainage bag and the drain line is connected to an opening having a venting line by means of which air can be introduced into the drain line, wherein a sterile filter is arranged in the drain line and/or in the venting line upstream of the opening and wherein a cut-off means is provided in the drain line upstream of the opening by means of which cut-off means the drain line can be cut off at times.

The terms “downstream” and “upstream” relate to the direction of flow of the dialyzate flowing off to the drain.

It is possible to completely empty the drain line due to the venting line opening into the drain line. In particular in CAPD, there are frequently undefined pressure conditions that depend on the height position of the patient relative to the drain so that the most unfavorable case can occur that the dialyzate contained in the drain line does not drain completely on the emptying of the patient, but rather remains therein or even flows back to the patient without being noticed, which can be accompanied by contaminations or infections that are naturally unwanted.

There is furthermore the possibility that the drain line has to bridge substantial height differences and a fluid column present in the drain line can cause a vacuum at the connection of the patient and thus at the abdomen. This unwanted effect should likewise be prevented.

It has proven advantageous if the length of the drain line is at least 2 m. This is the length that at a height difference causes a significant negative pressure in the peritoneum of the patient.

This is prevented by the venting line in accordance with the invention since the inflowing air effects the draining of the residual dialyzate present in the drain line into the outflow. The risk of contamination of the patient as well as risks to and the impairment of the well-being due to unwanted pressure conditions is thereby substantially reduced.

It must be pointed out at this point that the present invention is not restricted to one kind of treatment such as CAPD or APD, but rather covers any desired peritoneal dialysis treatments or machines and methods intended for this purpose. The present invention is, however, used primarily in CAPD.

The peritoneal dialysis machine is thus preferably a CAPD set.

In a further conceivable embodiment of the invention, the sterile filter in the venting line has hydrophobic properties.

Provision is furthermore preferably made that a further sterile filter is arranged in the drain line, preferably downstream of said opening of the venting line into the drain line.

The sterile filter or filters in the drain line has/have hydrophilic properties in a preferred embodiment of the invention.

The drain line and the venting line can be configured in one piece, i.e. can form a single part, or can also be connectable or connected to one another by means of a connector.

The drain line and/or the venting line are preferably designed as tubes.

Means for conveying air through the venting line into the drain line are preferably arranged in the venting line.

They can here be means to be actuated by a user or means that have a drive unit such as a compressor or another driven pump arrangement.

The means can be configured as bellows in a comparatively simple embodiment. The bellows can be formed in one piece with the venting line and can, for example, be formed from a film material or a tubing material.

The drain line and/or the venting line can have a port to which a container containing a disinfectant is arranged or can be arranged. It is thus possible to introduce a liquid or gaseous disinfectant into the drain line to particularly securely preclude germs, etc. which reach the patient in the most unfavorable case from colonizing the drain line.

The drain line can be provided with a connector for connecting the drain line to the peritoneal dialysis machine or to the patient catheter or to another part of the tube system. Said cut-off means can be located within the connector, but also outside and separate from the connector.

It is furthermore conceivable that the drain line is provided with a connector for connecting the drain line to the peritoneal dialysis machine, to the patient catheter, or to another part of the tubing system, wherein the connector is configured such that it is connectable or connected at one side to the peritoneal dialysis machine, to the patient catheter, or to another part of the tubing system and that it is connectable or connected at the other side to a connector already present at the drain line.

In this case, the drain line has a plurality of already used connectors at which a new, sterile connector is arranged before the draining of the consumed dialysis solution. This new connector can e.g. be connected to the patient catheter that thus only comes into contact with a sterile component, i.e. with the newly fitted connector.

Said cut-off means can, for example, be formed by a clamp or by a valve.

It is furthermore conceivable that the cut-off means is a one-way valve.

The present invention furthermore relates to a method for venting a drain line of a peritoneal dialysis machine in accordance with one of the claims 1 to 14, wherein the cut-off means is closed and wherein the venting of the drain line takes place subsequent thereto by means of the venting line, whereby the consumed dialysis solution is conducted into a drain which is not a bag or container.

It is conceivable that the venting takes place in that air, preferably environmental air, is conveyed at pressure into the drain line.

The conveying of the air can take place as described above manually or by means of a compressor.

To keep the drain line sterile in the event of its reuse, provision can be made that a sterilizing agent is introduced into the drain line before, during, or after the venting of the drain line. A conceivable variant here comprises the sterilizing agent being mixed into the air that is conveyed into the drain line. Alternatively to this, the sterilizing agent is added separately from the air that enters into the drain line.

The present invention furthermore relates to a tubing set for use in a peritoneal dialysis machine in accordance with one of the claims 1 to 14 and/or in a method in accordance with one of the claims 15 to 19, wherein the tubing set has a drain line having a connector for connecting to the peritoneal dialysis machine, to the patient catheter, or to another part of the tubing system and a venting line that is connected to the drain line at an opening such that air can be introduced from the venting line into the drain line, wherein air conveying means are provided that can be connected to the venting line via a connector or that form a component of the venting line.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further details and advantages of the invention will be described in more detail with reference to an embodiment shown in the drawing.

There are shown:

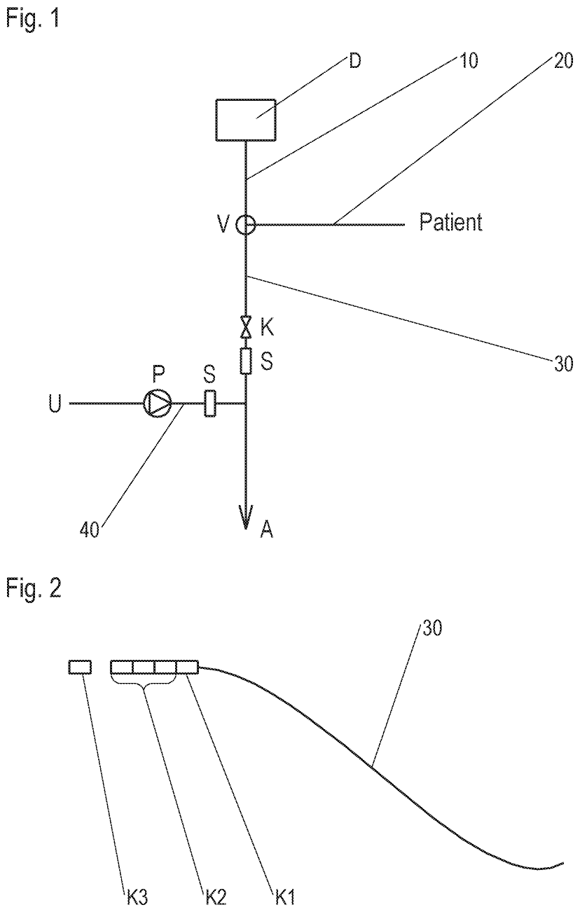

FIG. 1: a schematic view of a tubing set with a connected dialysis bag in accordance with the invention; and

FIG. 2: a view of a drainage tube with mutually connectable connectors.

A bag containing fresh dialysis solution is marked by reference symbol D in FIG. 1.

The distributor V is set during the filling phase such that the line 10 is in fluid communication with the line 20 so that fresh dialysis solution flows from the bag D into the peritoneum of the patient.

The clamp K in the drain line 30 is closed during this time.

After the elapse of a certain dwell time, the consumed dialysis solution is conducted through the line 20 into the drain line 30, for which purpose the distributor V is correspondingly adjusted such that there is a fluid communication between the lines 20, 30.

The clamp K is opened for this purpose and the consumed dialysis solution runs at least partly into the drain A such as into a sink, a shower tray, etc.

Since the pressure difference between the patient and the drain A caused by the height difference is possibly not sufficient to effect a complete emptying of the drain line 30, the venting line 40 opens into the drain line downstream of the clamp K. It is connected at its one end to the drain line 30 and at its other end to the environmental air U so that environmental air U can be sucked into the venting line 40 and can be conveyed from there into the drain lie 30.

To achieve the complete emptying of the drain line 30 downstream of the clamp K, the clamp K is closed and the compressor, bellows, etc. are put into operation, whereby air is introduced into the drain line 30 downstream of the clamp and the consumed dialyzate still remaining therein is completely conducted into the drain A.

Reference symbol S marks a sterile filter that ensures that no germs enter into the drain line 30 from the environmental air. It is located between the compressor, etc. and the opening of the line 40 into the line 30.

As can be seen from the Figure, a further sterile filter S is located in the drain line 30.

The control or the operation of the elements shown, in particular of the distributor V, of the clamp K, and of the compressor P, can take place manually or controlled by a control unit and corresponding actuating means.

FIG. 2 shows the drain line 30 that is provided at its end not facing the drain A with a connector that is e.g. connectable to the distributor or to the patient catheter, not shown. The patient catheter is a tubing section that leads into the peritoneum, that is fixedly connected to the patient, and that is connected via a connector to a tubing set to be able to introduce dialysis solution into the peritoneum and to conduct it out thereof.

The line 20 in accordance with FIG. 1 can, for example, comprise a first section that is formed by the patient catheter and a second section that leads to the distributor V, which is not shown in FIG. 1.

The connector K1 is the connector originally located at the drain line 30. The connectors K2 have been plugged on after one another, and indeed one respective fresh, sterile connector K2 per treatment. The connector K3 is a new, sterile connector that is used for the next draining of the dialysis solution from the peritoneum of the patient. It is plugged onto the last of the already present connectors K2 so that the patient catheter or another connector element that is connected to the connector K3 remains sterile.

The connectors can have a slit membrane that is opened in the connected state and thus releases a fluid passage.

The connection of the connectors can take place by a thread, a latch connection, etc.

1. A peritoneal dialysis machine having means for carrying out a peritoneal dialysis treatment, wherein the machine has a drain line which is connected or connectable to the patient catheter and through which consumed dialysis solution is conducted into a drain, characterized in that the drain line does not open into a drainage container; and in that the drain line is connected at an opening to a venting line by means of which the venting line can be vented, with a sterile filter being arranged in the drain line upstream of the opening and/or in the venting line and with a cut-off means by means of which the drain line can be cut off at times is provided in the drain line upstream of the opening.

2. A peritoneal dialysis machine in accordance with claim 1, characterized in that the peritoneal dialysis machine is a CAPD set.

3. A peritoneal dialysis machine in accordance with claim 1, characterized in that the sterile filter in the venting line has hydrophobic properties.

4. A peritoneal dialysis machine in accordance with claim 1, characterized in that a further sterile filter is provided in the drain line downstream of the opening.

5. A peritoneal dialysis machine in accordance with claim 1, characterized in that the sterile filter or filters in the drain line have hydrophilic properties.

6. A peritoneal dialysis machine in accordance with claim 1, characterized in that the drain line and the venting line are configured in one piece or are connectable or connected by means of a connector.

7. A peritoneal dialysis machine in accordance with claim 1, characterized in that means for conveying air through the venting line into the drain line are arranged in the venting line.

8. A peritoneal dialysis machine in accordance with claim 7, characterized in that the means are means to be actuated by a user or means that have a drive unit.

9. A peritoneal dialysis machine in accordance with claim 7, characterized in that the means are configured as bellows or as a compressor.

10. A peritoneal dialysis machine in accordance with claim 1, characterized in that the drain line and/or the venting line has a connection at which a container containing a disinfectant is arranged or can be arranged.

11. A peritoneal dialysis machine in accordance with claim 1, characterized in that the drain line is provided with a connector for connecting the drain line to the peritoneal dialysis machine; and in that the cut-off means is arranged in the connector.

12. A peritoneal dialysis machine in accordance with claim 1, characterized in that the drain line is provided with a connector for connecting the drain line to the peritoneal dialysis machine; and in that the connector is configured such that it is provided at the peritoneal dialysis machine at one side and at a connector already located at the drain line at the other side.

13. A peritoneal dialysis machine in accordance with claim 1, characterized in that the cut-off means are formed by a clamp or a valve.

14. A peritoneal dialysis machine in accordance with claim 1, characterized in that the cut-off means is a one-way valve.

15. A method for venting a drain line of a peritoneal dialysis machine in accordance with claim 1, characterized in that the cut-off means is closed first; and in that the venting of the drain line takes place subsequent thereto by means of the venting line, whereby the consumed dialysis solution is conducted into a drain which is not a drain container.

16. A method in accordance with claim 15, characterized in that the venting takes place in that air, preferably environmental air, is conveyed into the drain line at pressure.

17. A method in accordance with claim 16, characterized in that the conveying takes place manually or by means of a compressor.

18. A method in accordance with claim 15, characterized in that a sterilizing agent is introduced into the drain line before, during, or after the venting of the drain line.

19. A method in accordance with claim 18, characterized in that the sterilizing agent is mixed to the air that is conveyed into the drain line; or in that the sterilizing agent is added separately from the air that enters into the drain line.

20. A tubing set for use in a peritoneal dialysis machine in accordance with claim 1, wherein the tubing set has a drain line having a connector for connecting to the peritoneal dialysis machine, has a venting line that is connected to the drain line at an opening such that air can be introduced from the venting line into the drain line, wherein air conveying means are provided that can be connected to the venting line via a connector or that comprises a component of the venting line.
